(19) United States
(12) Patent Application Publication
(10) Pub. No.: US 2009/0220440 A1
(43) Pub. Date: Sep. 3, 2009

(54) Spray Composition for Topical Use for Treating and/or Preventing Herpes Simplex Labial Infections

(75) Inventor: Paolo Fabrizi, Monteriggioni (IT)

Correspondence Address: SALIWANCHIK LLOYD & SALIWANCHIK;A PROFESSIONAL ASSOCIATION, PO Box 142950, GAINESVILLE, FL, 32614, US

(73) Assignee: ISTITUTO FARMACOTERAPICO ITALIANO S.P.A., Rome (IT)

(21) Appl. No.: 12/282,283

(22) PCT Filed: Mar. 21, 2007
(86) PCT No.: PCT/EP2007/052689

§ 371 Date: Mar. 13, 2009

(30) Foreign Application Priority Data
Mar. 24, 2006 (IT) RM2006 A 000163

(51) Int. Cl. A61K 8/97; A61K 36/53; A61P 31/22

(52) U.S. Cl. 424/59; 424/725

(57) ABSTRACT

The present invention relates to a composition for the topical treatment of labial herpes lesions. The composition is based on preparations of officinal plants, with of anti-herpes simplex virus activity, anti-inflammatory and analgesic properties, and on natural purified components that have emollient, healing and covering effects on herpes eruptions. The composition contains essential oils of Mellissa officinalis and Lavandula angustifolia, glycyrrhizic acid, beta-glucan, D-pantenol and gelifiers.

### BACKGROUND OF THE INVENTION

Herpes simplex infections are caused by serotype 1 (HSV-1) in 80% of the cases and by serotype 2 (HSV-2), generally acquired subsequent to urogenital contacts, in the remaining cases. HSV-1 and HSV-2 are contracted by susceptible subjects, seronegative to anti-herpes antibodies, by close contact with infected saliva, mucosa and cutis. The primary infection may be symptomatic and asymptomatic. Symptomatic labial herpes infections develop with a papulous erythema rash that can develop into small vesicles filled with a clear liquid, with a high viral charge and into blisters generally localised along the outer margin of the lips. In some cases vesicles and blisters extend to the soft part of the palate, to the mouth and pharynx mucosa. The breaking of vesicles and blisters determines the forming of ulcerations accompanied by erythema, oedema and pain that make the consumption of food and beverages very difficult. Generally, ulcerations are cover with small scabs within 7-10 days. The fall of the scabs precedes the healing of ulcerations. In some cases, bacterial superinfections may occur. The virus migrates from the infection site to the spinal ganglia where it remains in the latency status without possibility to eradicate it and can be reactivated upon exposure to stimuli of various types (emotional and physical stress, exposure to UV sun radiations, fever, flu infections, menstruations) and cause relapses. In many patients subject to relapses, early symptoms localised in the eruption site and associated with pain, tingling, numbness and itching occur 12-36 hours prior to the appearance of vesicles and blisters (prodromic phase). In immuno-competent subjects, herpes infections are self-limiting. Therapy aims at preventing virus spreading and transmission and at shortening and opposing the symptomatology.

Oral or intravenous systemic antiviral treatment is limited to patients with greatly compromised immune system (HIV positive subjects, subjects with very evident AIDS, oncological patients or patients transplanted being in therapy with immunosuppressant drugs) in which herpes infection can generalise and take on particular gravity.

Prevention of herpes relapses is carried out by limiting and avoiding triggering factors, in order to prevent the onset of labial eruption or to reduce its gravity. The preventive systems regarded as the most effective are the application of topical protections containing sun filters and the taking of nutritional supplements such as lysine that has been proved to have a preventive valence when administered orally at high doses (1000 mg/die).

Topical therapies are the most followed ones by immunocompetent subjects and vary according to the evolutionary stage of the herpes infection.

In the initial steps, nucleosidic analogs that interfere on viral replication (acyclovir, pencyclovir) or inhibitors of virus cell fusion (docosanol) are used in creams. On the other hand, if the infection has progressed too much to proceed with antiviral therapy, treatments are prescribed for relieving pain, irritation and burn associated with the infection. In particular, oral analgesics (ibuprofen, paracetamol), local analgesics/anaesthetics (benzocaine, lidocaine, allantoin, phenol, menthol), skin protectors (kaolin, cocoa butter, glycerol, paraffin gel, zinc, oxide and acetate) that soften the scabs and relieve dryness, itching and the irritation that accompany the healing process are used.

The analysis of the prior art for local treatment systems for labial herpes eruptions reveal that an innovative therapeutic solution may be represented by a unique topical formulation based on a mixture of antiviral components, anti-inflammatory/analgesics, antimicrobics, emollients that favours the healing and covering of the lesions, and therefore applicable in the various infection steps for accelerating lesion healing process and reducing pain, irritation, risk of transmission and spreading of the infection and for reducing risk of bacterial superinfections.

### DESCRIPTION OF THE INVENTION

The object of the present invention is a topical composition based on preparations of officinal plants suitably selected and balanced for having optimum concentrations of active components for ensuring anti-herpes simplex virus of type 1 and 2 activity and indirect anti-inflammatory and analgesic properties useful for treating labial herpes infections. The same composition contains purified natural components with emollient, healing and covering activity that favour the healing process of herpes lesions and prevent the spreading of the infection process. The covering of vesicles and blisters consequent to the filming effect of the gel further improves the aesthetic appearance of the patient.

The composition is preferably in the form of spray gel, but other formulations are included in the instant invention. The composition comprises essential oil of Melissa officinalis and glycyrrhizic acid salts, preferably ammonium salts; in a preferred form the composition further comprises essential oil of Lavandula angustifolia and/or beta-glucan and/or D-pantenol (pro-vitamin B5).

Melissa officinalis is a plant whose essential oil contains components provided with activity of antivirus for herpes simplex (citral), antibacterial (geranyl and neral), analgesic (myrene) and antioxidant activity (isorientin, orientin, caffeic acid, chlorogenic acid). Essential oils of Melissa officinalis may be produced according to standard skill or are commercially available, i.e. from Esperis SpA, Milano, Italy. Titration and standardisation of the essential oil is among the ordinary skill.

Lavandula angustifolia is an officinal plant. The essential oil has anti-inflammatory, analgesic and antimicrobic properties. Moreover, it favours the healing of wounds. Essential oils of Lavandula angustifolia may be produced according to standard skill or are commercially available, i.e. from Esperis SpA, Milano, Italy. Titration and standardisation of the essential oil is among the ordinary skill.

Glycyrrhizic acid, present in the Glycyrrhiza glabra root extracts, exhibits anti-herpes simplex activity. Glycyrrhizic acid is commercially available for instance from Indena SpA, Milano, Italy.

Beta-glucan that can be extracted from yeast and algae and D-pantenol (pro-vitamin B5) are activators of the skin tissue regeneration. Beta-glucan activates the immune functions against viral, bacterial and fungus agents at a local level. Beta-glucan is commercially available for instance from Nutraceutica Srl, Monterenzio, Italy. D-pantenol is commercially available for instance from Res Pharma Srl, Trezzo sull'Adda, Italy.

In a preferred embodiment the composition of the invention contains, as regards the active components, from 0.5 to 2.0 % by weight of Melissa officinalis titrated and standardised essential oil, from 2.5 to 10% by weight of Lavandula angustifolia titrated and standardised essential oil, from 0.25% to 1% by weight of glycyrrhizic acid ammonium salt, from 0.25% to 1% by weight of beta-glucan, from 0.25% to 1% by weight of D-pantenol (pro-vitamin B5). The gel is stabilised at a pH between 4.5 and 5.5. The composition may further contain solubilisers, thickeners, stabilisers and gelifiers, i.e. carrageenans.

Chemical and physical filters provided with different protective index against UVA and UVB sunlight irradiation can be optionally added to the composition to reduce the frequency of recurrent outbreaks of local herpes simplex virus infections.

In a particularly preferred embodiment, the invention relates to a composition in the form of a spray gel that can be administered on herpes eruptions, containing the following quantities by weight of active components stabilised at pH 5.0:

Melissa officinalis titrated and standardised essential oil: 1%

Lavandula angustifolia titrated and standardised essential oil: 5%

glycyrrhizic acid ammonium salt: 0.5%

Beta-glucan: 0.5%

D-pantenol (pro-vitamin B5): 0.5%.

The composition is surprisingly effective, for the part consisting in the mixture of essential oils of Melissa officinalis and of glycyrrhizic acid, in inhibiting the “in vitro” replication of herpes simplex serotype 1 and 2. Moreover, associated to the essential oil of Lavandula angustifolia, to beta-glucan and to D-pantenol it has proved to be surprisingly effective in opposing the symptomatology induced by labial herpes infection and in favouring the healing of lesions induced by such infection.

The formulation may be produced following a standard pharmaceutical protocol for the preparation of bulk gel and its non-sterile bottling. The end product consists of the gel, distributed in dark glass bottles provided with special horizontal pump dispenser, vegetal extracted components associated with solubilising agents, thickeners, stabilisers and gelifiers of quality and in quantity approved for the pharmaceutical use.

The composition is advantageously used in the preventive and curative treatment of local type 1 and 2 herpes simplex virus infections.

The composition may be presented in form of spray container, labial stick, cream, etc.

The following non limiting examples illustrate the invention in greater detail:

### EXAMPLE 1

### Antiviral Activity and “in vitro” Cytotoxicity

Mixtures containing different percentages by weight of Melissa officinalis essential oil and of glycyrrhizic acid were assayed with conventional techniques for the “in vitro” measurement of the antiviral activity and of the cytotoxicity. Strains of herpes simplex type 1 (ATCC VR-260) and 2 (ATCC CR-734) were respectively replicated in cellular lines of Hep-2 and Vero cultivated, in EMEM supplemented with 10% FCS, at 37° C. with 5% CO₂. Suspensions of the single viruses at a multiplicity of infections (MOI) capable of producing a cytopathic effect (CPE) between 100% and 90% within 5-7 days, were pre-incubated with the mixture to be assayed and then inoculated in the specific cellular lines brought to flow into plate wells. The ability of the mixtures—at the final concentration ratios in micrograms/ml of Melissa officinalis essential oils/Glycyrrhizic acid ammonium salt corresponding to 100/50; 30/15; 10/5; 3/1,5; 1/0,5—to inhibit the viral replication based on the inhibition of the cytopathic effect (CPE) has been determined. In particular, the dilution of the source mixture was calculated by analysis of the linear regression, capable of inhibiting the cytopathic effect (IC50) by 50% as compared to the Hep-2 and Vero control cellular lines infected with the viral suspension not pre-incubated with the extracts. Equal concentrations of mixture extracts were added to the same non-infected cellular lines to determine the concentration of active component toxic in 50% of the cells (TC50).

The cytopathic effect and the cytotoxicity were measured as the capacity to reduce the metabolization by the mitochondrial enzymes of the tetrazolium dye MTS to a soluble colored formazan product that can be quantitate by spectrophotometric reading at 490/650 nm.

The mean dilution of the mixture that leads to a 50% inhibition (IC50) of the cytopathic effect induced by the various viruses was found to contain the following concentrations of active components:
Melissa officinalis essential oil: 10 micrograms/mL
Glycyrrhizic acid ammonium salt: 5 micrograms/mL

The optimal antiviral dilution of the mixture was not found to be cytotoxic.

### EXAMPLE 2

### Clinical Case

A 32 year old woman subject to frequent labial herpes relapses concurrent with her menstruation uses for the first time a therapeutic treatment by topical administration of a composition containing the following quantities by weight of active components stabilised at pH 5.0:

Melissa officinalis titrated and standardised essential oil: 1%

Lavandula angustifolia titrated and standardised essential oil: 5%

glycyrrhizic acid ammonium salt: 0.5%

Beta-glucan: 0.5%

D-pantenol (pro-vitamin B5): 0.5%.

The treatment is started in the prodromic phase. Smaller extension of herpes eruption, considerable reduction of pain, shorter duration of scabs followed by faster healing of herpes lesions are observed when compared to the previous recurrent infection episodes.

### EXAMPLE 3

### Clinical Case

A 35 year old woman, affected by labial herpes eruption further to a flu episode, starts upon the appearance of vesicles and blisters, the therapeutic treatment by topical administration of a composition containing the following quantities by weight of active components stabilised at pH 5.0:

Melissa officinalis titrated and standardised essential oil: 1%

Lavandula angustifolia titrated and standardised essential oil: 5%

glycyrrhizic acid ammonium salt: 0.5%

Beta-glucan: 0.5%

D-pantenol (pro-vitamin B5): 0.5%.

Healing is achieved in about four days and without having particular problems (itching, dryness, delay in the fall of scabs) and with perfect recovery of the integrity of the labial skin tissue affected by the herpes eruption.

1. A composition for topical use comprising at least the essential oil of Melissa officinalis and glycyrrhizic acid salts.

2. The composition according to claim 1 further comprising the essential oil of Lavandula angustifolia and/or beta-glucan and/or D-pantenol (pro-vitamin B5).

3. The composition according to claim 1, which is in the form of a buffered gel at pH between 4 and 6.

4. The composition according to claim 1, in the form of a spray.

5. The composition according to claim 1 comprising from 0.5 to 2% by weight of Melissa officinalis titrated and standardised essential oil, from 2.5 to 10% by weight of Lavandula angustifolia titrated and standardised essential oil, from 0.25% to 1% by weight of glycyrrhizic acid ammonium salt, from 0.25% to 1% by weight of beta-glucan, and from 0.25% to 1% by weight of D-pantenol (pro-vitamin B5).

6. The composition according to claim 5 further comprising solubilisers, thickeners, stabilisers and gelifiers.

7. The composition according to claim 1 comprising 1% by weight of Melissa officinalis titrated and standardised essential oil, 5% by weight of Lavandula angustifolia titrated and standardised essential oil, 0.5% by weight of glycyrrhizic acid ammonium salt, 0.5% by weight of beta-glucan and 0.5% by weight of D-pantenol.

8. The composition according to claim 1, further containing UV sunlight irradiation protective filters.

9. (canceled)

10. The composition according to claim 1, in the form of a medical device.

11. The composition according to claim 2, which is in the form of a buffered gel at pH between 4 and 6.

12. The composition according to claim 2, in the form of a spray.

13. A method for preventing or treating local type 1 and/or 2 herpes simplex virus infections, wherein said method comprises administering, to a subject in need of such prevention or treatment, a composition comprising at least the essential oil of Melissa officinalis and glycyrrhizic acid salts.

14. The method, according to claim 13, wherein said composition is applied topically.

15. The method, according to claim 13, wherein said composition further comprises the essential oil of Lavandula angustifolia and/or beta-glucan and/or D-pantenol (pro-vitamin B5).

16. The method, according to claim 13, wherein the composition is in the form of a buffered gel at pH between 4 and 6.

17. The method, according to claim 13, wherein the composition is applied as a spray.

18. The method, according to claim 13, wherein the composition comprises from 0.5 to 2% by weight of Melissa officinalis titrated and standardised essential oil, from 2.5 to 10% by weight of Lavandula angustifolia titrated and standardised essential oil, from 0.25% to 1% by weight of glycyrrhizic acid ammonium salt, from 0.25% to 1% by weight of beta-glucan, and from 0.25% to 1% by weight of D-pantenol (pro-vitamin B5).

19. The method, according to claim 13, wherein the composition further comprises solubilisers, thickeners, stabilizers and gelifiers.

20. The method, according to claim 13, wherein the composition comprises 1% by weight of Melissa officinalis titrated and standardised essential oil, 5% by weight of Lavandula angustifolia titrated and standardised essential oil, 0.5% by weight of glycyrrhizic acid ammonium salt, 0.5% by weight of beta-glucan and 0.5% by weight of D-pantenol.

21. The method, according to claim 13, wherein the composition further comprises UV sunlight irradiation protective filters.
